(19) United States
(12) Patent Application Publication
(10) Pub. No.: US 2008/0248469 A1
(43) Pub. Date: Oct. 9, 2008

(54) Methods for Identifying Nucleotides of Interest in Target Polynucleotides

(75) Inventor: Eugene G. Spier, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/838,639

(22) Filed: Aug. 14, 2007

Related U.S. Application Data
(60) Provisional application No. 60/822,623, filed on Aug. 16, 2006.

(51) Int. Cl. C12Q 1/68

(52) U.S. Cl. 435/6

(57) ABSTRACT

In some embodiments, the present teachings provide a method of identifying a nucleotide of interest in a target polynucleotide. In some embodiments, the method can comprise forming an amplification strand, wherein the amplification strand comprises a hairpinning end region; hybridizing the hairpinning end region of the amplification strand with a hairpinning region of a target polynucleotide, to form a self-complementary amplification product. After performing an extension reaction, wherein the hairpinning end region of the amplification strand is extended, an extended reaction product is formed. Detection of the extended reaction product can result in the identification of a nucleotide of interest in the target polynucleotide. Additional methods, as well as kits, are also provided.

### CROSS-REFERENCE TO RELATED APPLICATIONS

This application claims a priority benefit under 37 U.S.C. § 119(e) from U.S. Provisional Application No. 60/822,623, filed Aug. 16, 2006, the contents of which are incorporated herein by reference.

### FIELD

The present teachings relate to methods, compositions, and kits for determining the identity of nucleotide of interest in a target polynucleotide by forming a self-complementary amplification product.

### INTRODUCTION

Sequencing of the human genome, the HapMap project, and various technical advances allowing whole genome association studies, have lead to an ever expanding appreciation of the number of polymorphisms that are linked to medical conditions and phenotypic traits (Guttmacher and Collins, JAMA. 2005 Sep. 21;294(11):1399-402). Many single nucleotide polymorphisms (SNPs), multiple nucleotide polymorphisms (MNPs), copy number polymorphisms (CNPs), Loss of Heterozygosity (LOH), and large-scale polymorphisms will eventually move to the clinic, and become applicable in medically-relevant applications for patients. Improved approaches for elucidating the identity of polymorphic variations will be imperative to provide improved patient care in the area of clinical diagnostics.

The detection of the presence or absence of (or quantity of) one or more target nucleic acids in a sample or samples containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

Various methods of querying nucleic acids are known in the art, and include the polymerase chain reaction (“PCR”, for example see U.S. Pat. No. 4,965,188, U.S. Pat. No. 4,683,202, and U.S. Pat. No. 4,683,195), the oligonucleotide ligation assay (“OLA”, see for example U.S. Pat. No. 4,883,750, U.S. Pat. No. 5,242,794, U.S. Pat. No. 5,521,065, and U.S. Pat. No. 5,962,223), and various combinations of OLA and PCR (see for example U.S. Pat. No. 7,166,434, U.S. Pat. No. 7,097,980, U.S. Pat. No. 6,797,470, U.S. Pat. No. 6,506,594, U.S. Pat. No. 7,244,831, U.S. Pat. No. 7,083,917, U.S. Pat. No. 7,014,994, U.S. Pat. No. 6,852,487, U.S. Pat. No. 6,312,892, U.S. Pat. No. 6,268,148, U.S. Pat. No. 6,054,564, U.S. Pat. No. 6,027,889, U.S. Pat. No. 5,830,711, and U.S. Pat. No. 5,494,810). Various methods of cleaving detector probes, including detecting such cleavage products, are also known in the art (see for example U.S. Pat. No. 7,037,654 and U.S. Pat. No. 6,818,399). However, these methods can be difficult to perform, labor-intensive, expensive, and of limited through-put.

### SUMMARY

In some embodiments, the present teachings provide a method of identifying a nucleotide of interest in a target polynucleotide comprising; forming an amplification strand, wherein the amplification strand comprises a hairpinning end region; hybridizing the hairpinning end region of the amplification strand with a hairpinning region of a target polynucleotide; performing an extension reaction, wherein the hairpinning end region of the amplification strand is extended to form an extended reaction product; detecting the extended reaction product; and, identifying the target polynucleotide.

In some embodiments, the present teachings provide a method of identifying a nucleotide of interest in a target polynucleotide comprising; forming a reaction mixture comprising a first primer and a target polynucleotide, wherein the first primer comprises a hairpinning 5′ end region that is the same nucleotide sequence as a hairpinning region of the target polynucleotide, and a target specific portion, and wherein the hairpinning region of the target polynucleotide is adjacent to a nucleotide of interest; extending the first primer to form a first amplification strand; hybridizing a second primer to the first amplification strand; extending the second primer to form a second amplification strand; performing a PCR with the first primer and the second primer to form a PCR amplification product comprising the first amplification strand containing the first primer, and the second amplification strand containing the second primer, wherein the second amplification strand contains a hairpinning 3′ end region; hybridizing the hairpinning 3′ end region with the hairpinning region of the target polynucleotide, to form a self-complementary amplification product; providing a first ligation probe and a second ligation probe, wherein the first ligation probe comprises a first label and a first discriminating nucleotide, and wherein the second ligation probe comprises a second label and a second discriminating nucleotide; performing a hybridization reaction, wherein the first ligation probe, or the second ligation probe, hybridizes to the self-complementary amplification product; ligating the first ligation probe or the second ligation probe to the self-complementary ligation product to form a ligation product; detecting the ligation product; and, identifying the nucleotide of interest in the target polynucleotide.

In some embodiments, the present teachings provide a method of identifying a nucleotide of interest in a target polynucleotide comprising; forming a reaction composition comprising a target polynucleotide hybridized to a first primer; extending the first primer to form a first amplification strand; hybridizing a second primer to the first amplification strand, wherein the second primer comprises a target specific portion and a hairpinning 5′ end region that is the same nucleotide sequence as a hairpinning region of the target polynucleotide; extending the second primer to form a second amplification strand; performing a PCR with the first primer and the second primer to form a PCR amplification product comprising the first amplification strand containing the first primer, and the second amplification strand containing the second primer, wherein the second amplification strand contains a hairpinning 5′ end region; hybridizing the hairpinning 5′ end region with the hairpinning region of the target polynucleotide to form a self-complementary amplification product; providing a first ligation probe and a second ligation probe, wherein the first ligation probe comprises a first label and a first discriminating nucleotide, and wherein the second ligation probe comprises a second label and a second discriminating nucleotide; performing a hybridization reaction, wherein the first ligation probe, or the second ligation probe, hybridizes to the self-complementary amplification product; ligating the first ligation probe or the second ligation probe to the self-complementary ligation product to form a ligation product; detecting the ligation product; and, identifying the nucleotide of interest in the target polynucleotide.

Additional methods, as well as compositions and kits, are also provided.

### DRAWINGS

FIG. 1 depicts an illustrative schematic according to some embodiments of the present teachings.

FIG. 2 depicts an illustrative schematic according to some embodiments of the present teachings.

FIG. 3 depicts an illustrative schematic according to some embodiments of the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Some Definitions

As used herein, the term “hairpinning 5′ end region” refers to the 5′ end of a single-stranded nucleic acid that can hybridize with a hairpinning region of a target polynucleotide, or can hybridize with the complement to the hairpinning region of the target polynucleotide. The hybridization performed by a hairpinning 5′ end region is intramolecular, in that the hybridization is with a region of the same molecule.

As used herein, the term “hairpinning region of the target polynucleotide” refers to a sequence of bases, typically located between two PCR primer sites, which itself, or its complement, can hybridize with a hairpinning end region.

As used herein, the term “hairpinning 3′ end region” refers to the 3′ end of a single-stranded nucleic acid that can hybridize with a hairpinning region of a target polynucleotide, or can hybridize with the complement to the hairpinning region of the target polynucleotide. The hybridization performed by a hairpinning 3′ end region is intramolecular, in that the hybridization is with a region of the same molecule

As used herein, the term “hairpinning end region” is a general term that can refers to both 5′ hairpinning end regions, and/or 3′ hairpinning end regions, as will be apparent from the context

As used herein, the term “terminating nucleotide” refers to a nucleotide that can be added to a nucleic acid, but cannot itself be added to. For example, dideoxynucleotides are illustrative of a terminating nucleotide.

As used herein, the term “discriminating nucleotide” refers to a nucleotide present in a ligation probe, or present as a terminating nucleotide, which can hybridize to a nucleotide of interest, and hence result in the eventual identification of the nucleotide of interest in the target polynucleotide.

As used herein, the term “nucleotide of interest” refers to an unknown base in a target polynucleotide that is sought to be determined. For example, a single nucleotide polymorphism (SNP) is one illustration of a nucleotide of interest.

As used herein, the term “first amplification strand” refers to the result of a first primer extension reaction.

As used herein, the term “second amplification strand” refers to the result of a second primer extension reaction, which used as a template the first amplification strand.

As used herein, the terms “first primer” and “second primer” are intended only to orient the reader to the particular embodiment at hand, as will be clear from the context.

As used herein, the term “amplification strand” is a general term that is used to refer to a first amplification strand and/or a second amplification strand.

As used herein, the term “extension reaction” is a general term used to refer to the addition of at least one nucleotide to a nucleic acid. For example, a primer can be extended in an extension reaction to form an amplification strand. As another example, a terminating nucleotide can be extended in a single-base extension reaction to form a single-base extension product. As another example, a ligation probe can be ligated in an extension reaction to a self-complementary amplification product to form a ligation product.

As used herein, the term “extended reaction product” refers to the result of an extension reaction, and includes ligation products, amplification strands, and single-base extension products, as will be apparent from the context.

As used herein, the term “same nucleotide sequence as” is used to include the base adjacent to the nucleotide of interest. For example, a first primer comprises a hairpinning 5′ end region 5′CCAC is said to have the same nucleotide sequence as a hairpinning region of the target polynucleotide 5′TCCAC. The hairpinning 5′ end region, following amplification in a PCR, can comprise a template-independent A, thus accounting for the T at the 5′ end of the hairpinning region of the target polynucleotide. The term “nucleotide base” refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide” refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′-and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a nucleic acid include, but are not limited to, the structures:

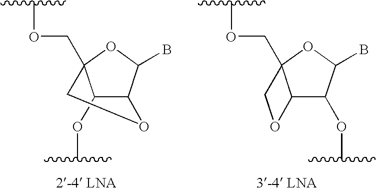

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

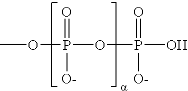

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog” refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into nucleic acid analogs in which the DNA/RNA phosphate ester and/or sugar phosphate ester backbone is replaced with a different type of internucleotide linkage. Exemplary nucleic acid analogs include, but are not limited to, peptide nucleic acids, in which the sugar phosphate backbone of the nucleic acid is replaced by a peptide backbone.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A nucleic acid may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

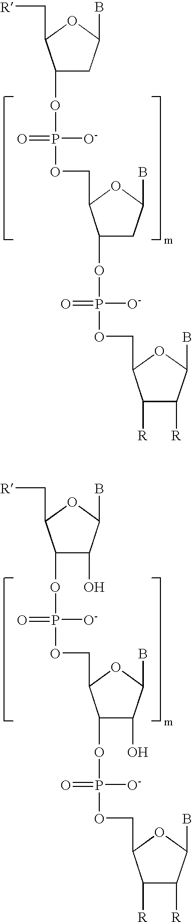

wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR″, and —NR″R″, where each R″ is independently (C₁-C₆) alkyl or (C₅-C1₄) aryl, or two adjacent Rs may be taken together to form a bond such that the ribose sugar is 2′,3′-didehydroribose, and each R′ may be independently hydroxyl or

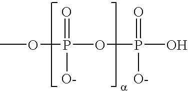

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A nucleic acid analog may comprise one or more lesions. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides. In various embodiments, sequences are complementary if they have 0%, 1%, 2%, or 3% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, and target-specific portions.

The term “primer” or “oligonucleotide primer” as used herein, refers to an oligonucleotide from which a primer extension product can be synthesized under suitable conditions. In certain embodiments, such suitable conditions comprise the primer being hybridized to a complementary nucleic acid and incubated in the presence of, for example, nucleotides, a polymerization-inducing agent, such as a DNA or RNA polymerase, at suitable temperature, pH, metal concentration, salt concentration, etc. In various embodiments, primers are 5 to 100 nucleotides long. In various embodiments, primers are 8 to 75, 10 to 60, 10 to 50, 10 to 40, or 10 to 35 nucleotides long.

The term “ligating” as used herein refers to the covalent joining of two nucleic acid ends. In various embodiments, ligation involves the covalent joining of a 3′ end of a first nucleic acid to a 5′ end of a second nucleic acid. In various embodiments, ligation results in a phosphodiester bond being formed between the nucleic acid ends. In various embodiments, ligation may be mediated by any enzyme, chemical, or process that results in a covalent joining of the nucleic acid ends. In certain embodiments, ligation is mediated by a ligase enzyme. Bond formation can include, without limitation, those created enzymatically by at least one DNA ligase or at least one RNA ligase, for example but not limited to, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, or the like, including but not limited to reversibly inactivated ligases (see, e.g., U.S. Pat. No. 5,773,258), and enzymatically active mutants and variants thereof. Other internucleotide linkages considered under “ligating” include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages. Ligating can also include chemical ligation, which can under appropriate conditions occur spontaneously, such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation. Ligation generally comprises at least one cycle of ligation, i.e., the sequential procedures of: hybridizing the target-specific portions of a first probe and a corresponding second probe to their respective complementary regions on the corresponding target nucleic acid sequences; ligating the 3′ end of the upstream probe with the 5′ end of the downstream probe to form a ligation product; and denaturing the nucleic acid duplex to release the ligation product from the ligation product:target nucleic acid sequence duplex. The ligation cycle may or may not be repeated, for example, without limitation, by thermocycling the ligation reaction to amplify the ligation product using ligation probes. Descriptions of these techniques can be found in, among other places, U.S. Pat. Nos. 5,185,243 and 6,004,826, 5,830,711, 6,511,810, 6,027,889; published European Patent Applications EP 320308 and EP 439182; Published PCT applications WO 90/01069, WO 01/57268, WO0056927A3, WO9803673A1, WO200117329, Landegren et al., Science 241:1077-80 (1988), and Day et al., Genomics, 29(1): 152-162 (1995).

The term “target polynucleotide” as used herein refers to an RNA or DNA that has been selected for detection. Exemplary RNAs include, but are not limited to, mRNAs, tRNAs, snRNAs, rRNAs, retroviruses, small non-coding RNAs, microRNAs, polysomal RNAs, pre-mRNAs, intronic RNA, and viral RNA. Exemplary DNAs include, but are not limited to, genomic DNA, plasmid DNA, phage DNA, nucleolar DNA, mitochondrial DNA, chloroplast DNA, cDNA, synthetic DNA, yeast artificial chromosomal DNA (“YAC”), bacterial artificial chromosome DNA (“BAC”), other extrachromosomal DNA, and primer extension products. Exemplary methods for detecting short nucleic acids, e.g., using stem-loop primers and/or short primers, can be found, e.g., in U.S. Patent Publication No. US 2005/0266418 to Chen et al., and U.S. Patent Publication No. US 2006/0057595 to Lao et al.

The term “sample” as used herein refers to any sample that is suspected of containing a target analyte and/or a target polynucleotide. Exemplary samples include, but are not limited to, prokaryotic cells, eukaryotic cells, tissue samples, viral particles, bacteriophage, infectious particles, pathogens, fungi, food samples, bodily fluids (including, but not limited to, mucus, blood, plasma, serum, urine, saliva, and semen), water samples, and filtrates from, e.g., water and air.

As used herein, the term “amplification” refers to any method for increasing the amount of a target nucleic acid, or amount of signal indicative of the presence of a target nucleic acid, wherein the amplification comprises a detector probe whose cleavage results in a free zip-code. Illustrative methods include the polymerase chain reaction (PCR), rolling circle amplification (RCA), helicase dependant amplification (HDA), Nucleic Acid Sequence Based Amplification (NASBA), ramification amplification method (RAM), recombinase-polymerase amplification (RPA), and others.

As used herein, the term “label” refers to detectable moieties that can be attached to detector probes to thereby render the molecule detectable by an instrument or method. For example, a label can be any moiety that: (i) provides a detectable signal; (ii) interacts with a second label to modify the detectable signal provided by the first or second label; or (iii) confers a capture function, e.g. hydrophobic affinity, antibody/antigen, ionic complexation. The skilled artisan will appreciate that many different species of labels can be used in the present teachings, either individually or in combination with one or more different labels. Exemplary labels include, but are not limited to, fluorophores, radioisotopes, Quantum Dots, chromogens, Sybr Green™, enzymes, antigens including but not limited to epitope tags, heavy metals, dyes, phosphorescence groups, chemiluminescent groups, electrochemical detection moieties, affinity tags, binding proteins, phosphors, rare earth chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Neb.), electrochemiluminescence labels, including but not limited to, tris(bipyridal) ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

As used herein, the term “fluorophore” refers to a label that comprises a resonance-delocalized system or aromatic ring system that absorbs light at a first wavelength and emits fluorescent light at a second wavelength in response to the absorption event. A wide variety of such dye molecules are known in the art, as described for example in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, EP 050684, and U.S. Pat. Nos. 5,750,409 and 5,066,580. Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In some embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.)

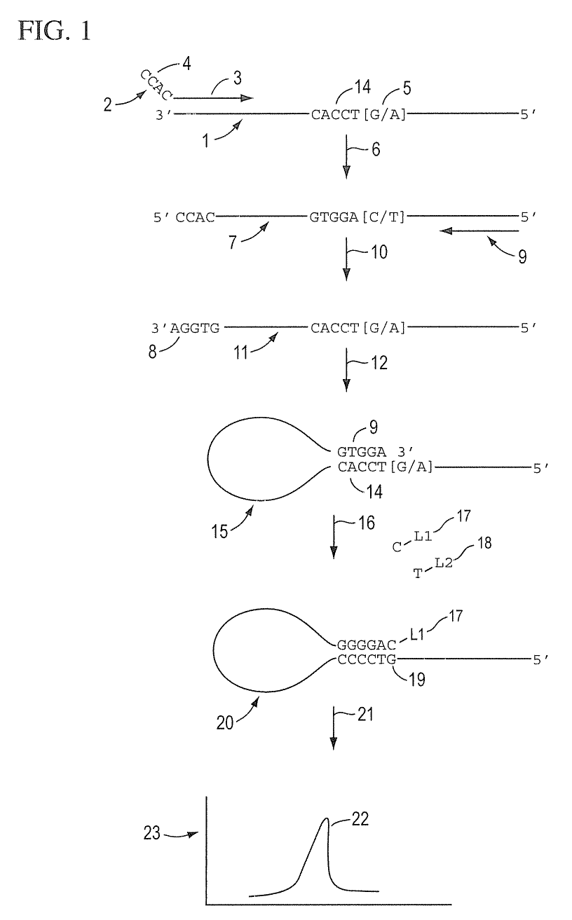

In some embodiments, the present teachings provide a method of identifying a nucleotide of interest in a target polynucleotide. One illustrative embodiment is depicted in FIG. 1. Here, a target polynucleotide (1) is shown in a reaction mixture hybridized with a first primer (2), wherein the first primer comprises a hairpinning 5′ end region (CCAC, 4) that is the same nucleotide sequence as a hairpinning region of the target polynucleotide (CCAC, 14). The hairpinning region of the target polynucleotide (14) is adjacent to the nucleotide of interest (5, a single nucleotide polymorphism (SNP) for example, depicted here as a G or an A). The first primer (2) also comprises a target specific portion (3) After extension (6) of the first primer, a first amplification strand is formed (7), to which a second primer (9) can hybridize and itself be extended (10), thus forming a second amplification strand (11). Cycling the temperature a number of times results in the performance of a polymerase chain reaction (PCR). The PCR results in the formation of a PCR amplification product. The PCR amplification product comprises the first amplification strand containing the first primer (7), and the second amplification strand containing the second primer (11). The second amplification strand contains a hairpinning 3′ end region (8), which results from the complementary polymerization of the hairpinning 5′ end region (4) of the first primer (2) in the first amplification strand (7). This hairpinning 3′ end region (8) can further contain an A at its 3′ end, which can result from the template-independent A addition that some DNA polymerases exhibit in PCR. Following reduced temperature (12), the hairpinning 3′ end region can hybridize with the hairpinning region of the target polynucleotide (14), to form a self-complementary amplification product (15). Labeled terminating nucleotides (17, a dideoxy-C comprising a label L1, and 18, a dideoxy-T comprising a label L2) can be provided, and a single-base extension reaction performed (16). The single-base extension reaction (16) results in the formation of a single-base extension product (20), which here contains the dideoxy-C comprising the label L1 (17), since there was a G at the nucleotide of interest (19). The single-base extension product (20) can be detected using a mobility dependent analysis technique. For example, capillary electrophoresis (21) can be used to detect the distinct size (22) and color of the single-base extension product in an electropherogram (23), thus allowing for the identification of the nucleotide of interest in the target polynucleotide.

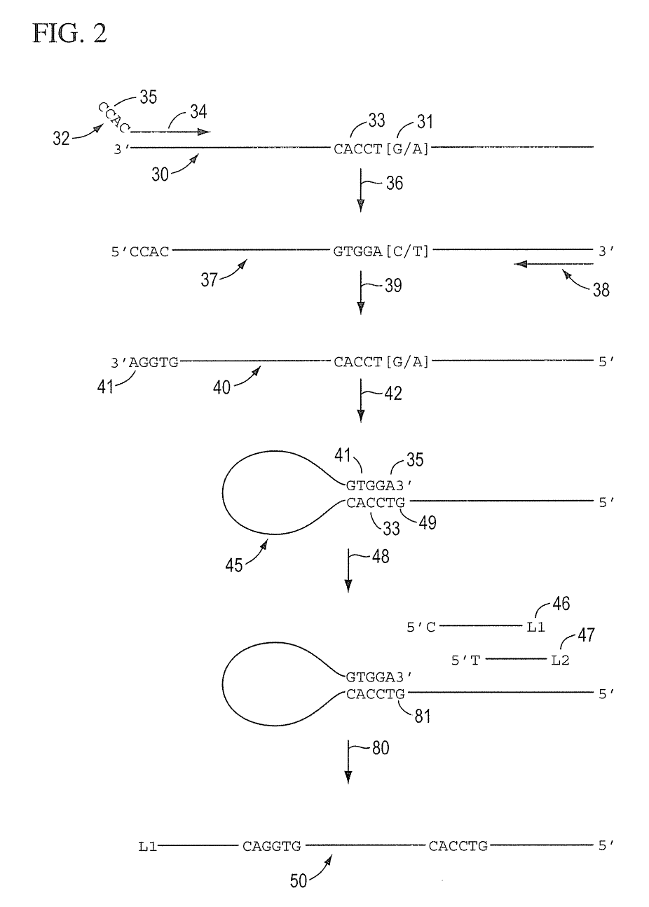

Another illustrative embodiment is depicted in FIG. 2. Here, a target polynucleotide (30) is shown in a reaction mixture hybridized with a first primer (32). The first primer comprises a hairpinning 5′ end region (CCAC, 35) that is the same nucleotide sequence as a hairpinning region of the target polynucleotide (TCCAC, 33). The hairpinning region of the target polynucleotide (33) is adjacent to a nucleotide of interest (31, a G or an A). The first primer (32) also comprises a target specific portion (34). After extension (36) of the first primer (32), a first amplification (37) is formed. A second primer (38) can hybridize to the first amplification strand (37) and be extended (39), thus forming a second amplification strand (40). Temperature cycling a number of times results in the performance of a PCR, which results in the formation of a PCR amplification product. The PCR amplification product comprises the first amplification strand containing the first primer (37), and the second amplification strand containing the second primer (40). The second amplification strand contains a hairpinning 3′ end region (41), which results from the complementary polymerization of the hairpinning 5′ end region (35) of the first primer (32) in the first amplification strand (37). This hairpinning 3′ end region (41) can contain an A at its 3′ end, which results from the template-independent A addition that some DNA polymerases exhibit in PCR. Following reduced temperature (42), the hairpinning 3′ end region (41) can hybridize with the hairpinning region of the target polynucleotide (33), to form a self-complementary amplification product (45). Labeled ligation probes ((46), a first ligation probe comprising a 5° C. and a label L1, and (47), a second ligation probe comprising a label a 5′T and a label L2) can be provided (48), and a ligation reaction performed (80). The ligation reaction (80) results in the formation of a ligation product (50), which here contains the first ligation probe (46) comprising the label L1, since there is a G at the nucleotide of interest (81). The ligation product (50) can be detected using any variety of techniques, including a mobility dependent analysis technique such as capillary electrophoresis, and the identification of the nucleotide of interest in the target polynucleotide determined.

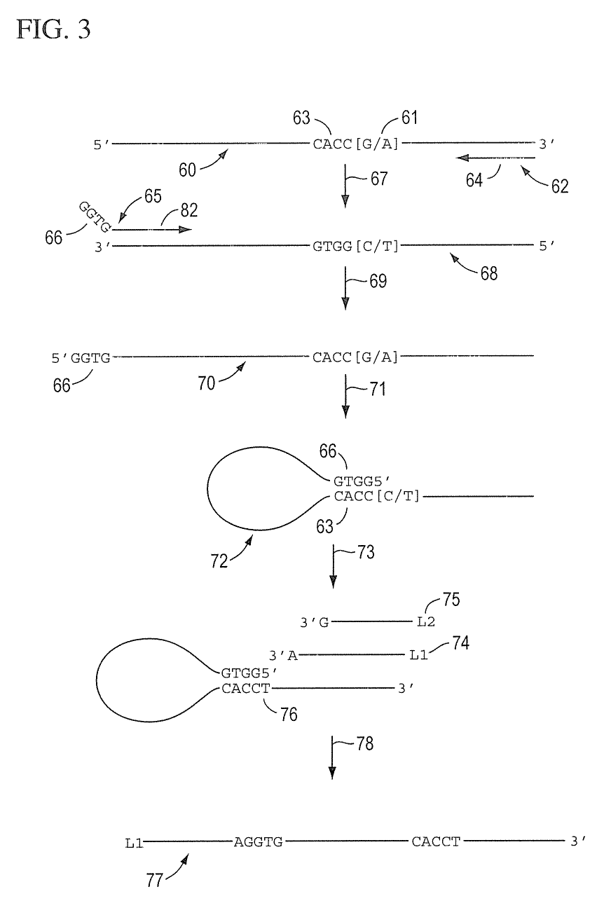

Another illustrative embodiment is depicted in FIG. 3. Here, a target polynucleotide (60) is shown in a reaction mixture, including a hairpinning region of the target polynucleotide (63, CACC), and a nucleotide of interest (61). The target polynucleotide (60) is hybridized with a first primer (62), wherein the first primer comprises a target specific portion (64). After extension (67) of the first primer (62), a first amplification strand (68) is formed, to which a second primer (65) can hybridize. The second primer (65) comprises a target specific portion (82) and a hairpinning 5′ end region (GGTG, 66) that is the same nucleotide sequence as the complement of the hairpinning region of the target polynucleotide (CAAC, 63, shown here as GTGG in the first amplification strand (68)). The second primer (65) can be extended (69), thus forming a second amplification strand (70). Temperature cycling a number of times results in the performance of a PCR, which results in the formation of a PCR amplification product. The primary PCR amplification product comprises the first amplification strand containing the first primer and the polymerized complement of the 5′ end of the second primer (not shown), and the second amplification strand containing the second primer (70). The second amplification strand contains a hairpinning 5′ end region (66), which results from the incorporation of the second primer (65) in the second amplification strand (70). Following reduced temperature (71), the hairpinning 5′ end region (66) can hybridize with the hairpinning region of the target polynucleotide (63), to form a self-complementary amplification product (72). Labeled ligation probes ((74), a first ligation probe comprising a 3′A and a label L1, and (75), a second ligation probe comprising a 3′G and a label L2) can be provided (73), and a ligation reaction performed (78). The ligation reaction (78) results in the formation of a ligation product (77), which here contains the first ligation probe (74) comprising the label L1, since there is a T at the nucleotide of interest (76), corresponding to an A at the nucleotide of interest (61) in the target polynucleotide (60). The ligation product (77) can be detected using any of a variety of techniques, including mobility dependent analysis techniques such as capillary electrophoresis, and the identification of the nucleotide of interest in the target polynucleotide determined.

It will be appreciated that the length of the hairpinning regions can vary according to the particular experimental context. The somewhat short four and five-mer sequences depicted in FIGS. 1-3 are present for clarity of presentation. In an experimental setting, hairpinning regions of 5-10, 10-30, and 10-20 nucleotides are more likely to be employed. Illustrative teachings for addressing cross-hybridization and minimizing unwanted interactions, as well as general teachings for related sequence length to melting temperature applicable to the present teachings, can be found for example in Biochimie 67, 685-695, Blommers et al. (1989) Biochemistry 28, 7491-7498, Antao et al. (1991) Nucleic Acids Res. 19, 5901-5905, Antao et al. (1991) Nucleic Acids Res. 20, 819-824, Senior et al. (1988) Proc. Natl. Acad. Sci. USA 85, 6242-6246, Proc. Nati. Acad. Sci. USA 95, 1460-1465, Allawi, H T & SantaLucia, J Jr. (1997), Biochemistry 36, 10581-10594, Allawi, H T & SantaLucia, J Jr. (1998), Biochemistry 37, 2170-2179, Allawi, H T & SantaLucia, J Jr. (1998) , Nucleic Acids Research 26, 2694-270, Allawi, H T & SantaLucia, J Jr. (1998) Biochemistry 37, 9435-9444, and Peyret, N, Seneviratne, P A, Allawi, H T & SantaLucia, J Jr. (1999), Biochemistry 38, 3468-3477.

One of skill in the art will appreciate that any number of routine reagents can be employed in the context of the present teachings. For example, polymerase, and enzymes generally, are commercially available from a variety of sources, including Applied Biosystems, New England Biolabs, and other vendors. Also readily available are nucleotides needed for PCR, primers, reaction buffers and the like. Further, it will be appreciated that the primers and probes discussed in the present teachings can comprise natural nucleotides, as well as various analogs readily available to one of skill in the art (see above definitions), including for example locked nucleic acid (LNA). The reaction products resulting from the methods of the present teachings can be detected using any of a variety of procedures, including mobility dependent analysis techniques such as capillary electrophoresis, mass spec, and HPLC.

Methods of performing PCR are routine in molecular biology, with further description being available, for example, in U.S. Pat. No. 4,638,202. Methods of performing single-base extension reaction are also routine, with further description being available, for example, in U.S. Pat. No. 5,856,092. Ligation reactions, for example using labeled probes, are also routine, with further description being available, for example, U.S. Pat. No. 4,883,750, U.S. Pat. No. 5,470,705, and U.S. Pat. No. 6,797,470. Labels, such as florophores, that can be used in the context of the present teachings can be found further described, for example, in U.S. Pat. No. 4,855,225, U.S. Pat. No. 6,649,598, U.S. Pat. No. 5,366,860, and U.S. Pat. No. 5,188,934. Descriptions of mobility dependant analysis techniques, such as capillary electrophoresis, can be found for example in U.S. Pat. No. 5,468,365, U.S. Pat. No. 5,384,024, and U.S. Pat. No. 5,290,418. Further routine molecular biology techniques readily available to one of skill in the art of molecular biology that can be applied in the context of the present teachings can be found in Sambrook et al., Molecular Cloning, 3rd Edition.

Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

For example, some embodiments the present teachings comprise a kit comprising a first primer, wherein the first primer comprises a target specific portion and a hairpinning end region; a second primer; and, a terminating nucleotide. In some embodiments, the kit can further comprise a polymerase, dNTPs, PCR buffer, or combinations thereof.

In some embodiments, the present teachings provide a kit for identifying a nucleotide of interest in a target polynucleotide comprising; a first primer, wherein the first primer comprises a target specific portion and a hairpinning end region; a second primer; and, a first ligation probe and a second ligation probe. In some embodiments, the kit can further comprise a polymerase, dNTPs, PCR buffer, ligase, or combinations thereof.

While the present teachings have been described in terms of these exemplary embodiments, the skilled artisan will readily understand that numerous variations and modifications of these exemplary embodiments are possible without undue experimentation. All such variations and modifications are within the scope of the present teachings.

Further, the foregoing description details certain preferred embodiments of the invention and describes the best mode contemplated by the inventors. It will be appreciated, however, that no matter how detailed the foregoing may appear in text, the present teachings may be practiced in many ways and should be construed in accordance with the appended claims and any equivalents thereof.

We claim:

1. A method of identifying a nucleotide of interest in a target polynucleotide comprising;
forming a reaction mixture comprising a first primer and a target polynucleotide, wherein the first primer comprises a hairpinning 5′ end region that is the same nucleotide sequence as a hairpinning region of the target polynucleotide, and a target specific portion, and wherein the hairpinning region of the target polynucleotide is adjacent to a nucleotide of interest;
extending the first primer to form a first amplification strand;
hybridizing a second primer to the first amplification strand;
extending the second primer to form a second amplification strand; performing a PCR with the first primer and the second primer to form a PCR amplification product comprising the first amplification strand containing the first primer, and the second amplification strand containing the second primer, wherein the second amplification strand contains a hairpinning 3′ end region;
hybridizing the hairpinning 3′ end region with the hairpinning region of the target polynucleotide, to form a self-complementary amplification product;
providing a first labeled terminating nucleotide and a second labeled terminating nucleotide, wherein the first labeled terminating nucleotide comprises a first label and a first discriminating nucleotide, and wherein the second labeled terminating nucleotide comprises a second label and a second discriminating nucleotide;
performing a single base extension reaction to form a single-base extension product;
detecting the single base extension product; and,
identifying the nucleotide of interest in the target polynucleotide.

2. The method according to claim 1 wherein the detecting comprises a mobility dependent analysis technique.

3. The method according to claim 1 wherein the mobility dependent analysis technique is capillary electrophoresis.

4. The method according to claim 1 wherein the first label, the second label, or both the first label and the second label are florophores.

5. The method according to claim 1 wherein the first terminating nucleotide, the second terminating nucleotide, or both the first terminating nucleotide and the second terminating nucleotide are dideoxynucleotides.

6. The method according to claim 1 wherein the hairpinning 3′ end region of the second amplification strand further comprises a template-independent A, and wherein the nucleotide adjacent to the nucleotide of interest is a T.

7. A method of identifying a nucleotide of interest in a target polynucleotide comprising;
forming a reaction mixture comprising a first primer and a target polynucleotide, wherein the first primer comprises a hairpinning 5′ end region that is the same nucleotide sequence as a hairpinning region of the target polynucleotide, and a target specific portion, and wherein the hairpinning region of the target polynucleotide is adjacent to a nucleotide of interest;
extending the first primer to form a first amplification strand;
hybridizing a second primer to the first amplification strand;
extending the second primer to form a second amplification strand;
performing a PCR with the first primer and the second primer to form a PCR amplification product comprising the first amplification strand containing the first primer, and the second amplification strand containing the second primer, wherein the second amplification strand contains a hairpinning 3′ end region;
hybridizing the hairpinning 3′ end region with the hairpinning region of the target polynucleotide, to form a self-complementary amplification product;
providing a first ligation probe and a second ligation probe, wherein the first ligation probe comprises a first label and a first discriminating nucleotide, and wherein the second ligation probe comprises a second label and a second discriminating nucleotide;
performing a hybridization reaction, wherein the first ligation probe, or the second ligation probe, hybridizes to the self-complementary amplification product;
ligating the first ligation probe or the second ligation probe to the self-complementary ligation product to form a ligation product;
detecting the ligation product; and,
identifying the nucleotide of interest in the target polynucleotide.

8. The method according to claim 7 wherein the detecting comprises a mobility dependent analysis technique.

9. The method according to claim 7 wherein the mobility dependent analysis technique is capillary electrophoresis.

10. The method according to claim 7 wherein the first label, the second label, or both the first label and the second label are florophores.

11. The method according to claim 7 wherein the first ligation probe comprises a 5′ end, and wherein the 5′ end of the first ligation probe comprises the discriminating nucleotide of the first ligation probe, wherein the second ligation comprises a 5′ end, and wherein the 5′ end of the second ligation probe comprises the discriminating nucleotide of the second ligation probe.

12. A method of identifying a nucleotide of interest in a target polynucleotide comprising;
forming a reaction composition comprising a target polynucleotide hybridized to a first primer;
extending the first primer to form a first amplification strand;
hybridizing a second primer to the first amplification strand, wherein the second primer comprises a target specific portion and a hairpinning 5′ end region that is the same nucleotide sequence as a hairpinning region of the target polynucleotide;
extending the second primer to form a second amplification strand;
performing a PCR with the first primer and the second primer to form a PCR amplification product comprising the first amplification strand containing the first primer, and the second amplification strand containing the second primer, wherein the second amplification strand contains a hairpinning 5′ end region;
hybridizing the hairpinning 5′ end region with the hairpinning region of the target polynucleotide to form a self-complementary amplification product;
providing a first ligation probe and a second ligation probe, wherein the first ligation probe comprises a first label and a first discriminating nucleotide, and wherein the second ligation probe comprises a second label and a second discriminating nucleotide;
performing a hybridization reaction, wherein the first ligation probe, or the second ligation probe, hybridizes to the self-complementary amplification product;
ligating the first ligation probe or the second ligation probe to the self-complementary ligation product to form a ligation product;
detecting the ligation product; and,
identifying the nucleotide of interest in the target polynucleotide.

13. The method according to claim 11 wherein the detecting comprises a mobility dependent analysis technique.

14. The method according to claim 11 wherein the mobility dependent analysis technique is capillary electrophoresis.

15. The method according to claim 11 wherein the first label, the second label, or both the first label and the second label are florophores.

16. The method according to claim 11 wherein the first ligation probe comprises a 3′ end, and wherein the 3′ end of the first ligation probe comprises the discriminating nucleotide of the first ligation probe, wherein the second ligation comprises a 3′ end, and wherein the 3′ end of the second ligation probe comprises the discriminating nucleotide of the second ligation probe.

17. A method of identifying a nucleotide of interest in a target polynucleotide comprising;
forming an amplification strand, wherein the amplification strand comprises a hairpinning end region;
hybridizing the hairpinning end region of the amplification strand with a hairpinning region of a target polynucleotide;
performing an extension reaction, wherein the hairpinning end region of the amplification strand is extended to form an extended reaction product;
detecting the extended reaction product; and,
identifying the target polynucleotide.

18. The method according to claim 17 wherein the detecting comprises a mobility dependent analysis technique.

19. The method according to claim 18 wherein the mobility dependent analysis technique is capillary electrophoresis.

20. The method according to claim 18 wherein the extension reaction comprises a single base extension reaction, said method further comprising providing a first labeled terminating nucleotide and a second labeled terminating nucleotide, wherein the first labeled terminating nucleotide comprises a first label and a first discriminating nucleotide, and wherein the second labeled terminating nucleotide comprises a second label and a second discriminating nucleotide;
performing a single base extension reaction to form a single-base extension product;
detecting the single base extension product; and,
identifying the nucleotide of interest in the target polynucleotide.

21. The method according to claim 20 wherein the first label, the second label, or both the first label and the second label are florophores.

22. The method according to claim 20 wherein the first terminating nucleotide, the second terminating nucleotide, or both the first terminating nucleotide and the second terminating nucleotide are dideoxynucleotides.

23. The method according to claim 18 wherein the extension reaction comprises a ligation reaction, said method further comprising providing a first ligation probe and a second ligation probe, wherein the first ligation probe comprises a first label and a first discriminating nucleotide, and wherein the second ligation probe comprises a second label and a second discriminating nucleotide;
performing a hybridization reaction, wherein the first ligation probe, or the second ligation probe, hybridizes to the self-complementary amplification product;
ligating the first ligation probe or the second ligation probe to the self-complementary ligation product to form a ligation product;
detecting the ligation product; and,
identifying the nucleotide of interest in the target polynucleotide.

24. The method according to claim 23 wherein the first label, the second label, or both the first label and the second label are florophores.

25. The method according to claim 23 wherein the first ligation probe comprises a 3′ end, and wherein the 3′ end of the first ligation probe comprises the discriminating nucleotide of the first ligation probe, wherein the second ligation comprises a 3′ end, and wherein the 3′ end of the second ligation probe comprises the discriminating nucleotide of the second ligation probe.

26. The method according to claim 23 wherein the first ligation probe comprises a 5′ end, and wherein the 5′ end of the first ligation probe comprises the discriminating nucleotide of the first ligation probe, wherein the second ligation comprises a 5′ end, and wherein the 5′ end of the second ligation probe comprises the discriminating nucleotide of the second ligation probe.

27. A kit for identifying a nucleotide of interest in a target polynucleotide comprising;
a first primer, wherein the first primer comprises a target specific portion and a hairpinning end region;
a second primer; and,
a terminating nucleotide.

28. The kit according to claim 27 further comprising a polymerase, dNTPs, PCR buffer, or combinations thereof.

29. A kit for identifying a nucleotide of interest in a target polynucleotide comprising;
a first primer, wherein the first primer comprises a target specific portion and a hairpinning end region;
a second primer; and,
a first ligation probe and a second ligation probe.

30. The kit according to claim 30 further comprising a polymerase, dNTPs, PCR buffer, ligase, or combinations thereof.
